(19) United States
(12) Patent Application Publication
(10) Pub. No.: US 2009/0196905 A1
(43) Pub. Date: Aug. 6, 2009

(54) STABILIZATION OF MITOCHONDRIAL MEMBRANES IN OCULAR DISEASES AND CONDITIONS

(76) Inventors: Lon T. Spada, Walnut, CA (US); Wendy M. Blanda, Tustin, CA (US); Marianne M. Do, Orange, CA (US); Scott M. Whitcup, Laguna Hills, CA (US); Patrick M. Hughes, Aliso Viejo, CA (US); Michael R. Robinson, Irvine, CA (US)

Correspondence Address: ALLERGAN, INC., 2525 DUPONT DRIVE, T2-7H, IRVINE, CA, 92612-1599, US

(21) Appl. No.: 12/026,736

(22) Filed: Feb. 6, 2008

(51) Int. Cl. A61F 2/00; A61K 38/08

(52) U.S. Cl. 424/427; 514/15

(57) ABSTRACT

Methods of treating ocular diseases and conditions using biodegradable ocular implants containing cyclosporine to inhibit mitochondrial permeability transition pore formation are disclosed.

### BACKGROUND

The present invention relates to drug delivery systems (eg implants), as well as to methods for treating ocular conditions with extended or sustained drug release of an active therapeutic agent from a biodegradable intraocular implant. In particular the present invention relates to implants and methods for treating a posterior ocular condition by implanting into an ocular region or site such as the vitreous a drug delivery system comprising an extended release, active agent incorporating, bioerodible implant.

The mammalian eye comprises an outer covering including the sclera (the tough white portion of the exterior of the eye) and the cornea, the clear outer portion covering the pupil and iris. In a medial cross section, from anterior to posterior, the eye comprises features including: the cornea, anterior chamber (a hollow space filled with a watery clear fluid called the aqueous humor and bounded by the cornea in the front and the lens in the posterior direction), iris (a curtain-like structure that can open and close in response to ambient light), lens, posterior chamber (filled with a viscous fluid called the vitreous humor), retina (the innermost coating of the back of the eye comprised of light-sensitive neurons), choroid (and intermediate layer providing blood vessels to the cells of the eye), and the sclera. In the human eye the posterior chamber comprises approximately ⅔ of the inner volume of the eye, while the anterior chamber and its associated features (lens, iris etc.) comprise about ⅓ of the eye's volume.

The delivery of therapeutic agents to the anterior surface of the eye is routinely carried out by topical means such as eye drops. However, the delivery of therapeutic agents to the interior or back of the eye, even the inner portions of the cornea, presents unique challenges. Drugs are available that may be used to treat diseases of the posterior segment of the eye, including pathologies of the posterior sclera, the uveal tract, the vitreous, the choroid, retina and optic nerve head (ONH).

An ocular condition can include a disease, aliment or condition which affects or involves the eye or one of the parts or regions of the eye. Broadly speaking the eye includes the eyeball and the tissues and fluids which constitute the eyeball, the periocular muscles (such as the oblique and rectus muscles) and the portion of the optic nerve which is within or adjacent to the eyeball. An anterior ocular condition is a disease, ailment or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves, the conjunctiva, the cornea, the conjunctiva, the anterior chamber, the iris, the posterior chamber (behind the retina but in front of the posterior wall of the lens capsule), the lens or the lens capsule and blood vessels and nerve which vascularize or innervate an anterior ocular region or site. A posterior ocular condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site.

Thus, a posterior ocular condition can include a disease, ailment or condition, such as for example, macular degeneration (such as non-exudative age related macular degeneration and exudative age related macular degeneration); choroidal neovascularization; acute macular neuroretinopathy; macular edema (such as cystoid macular edema and diabetic macular edema); Behcet's disease, retinal disorders, diabetic retinopathy (including proliferative diabetic retinopathy); retinal arterial occlusive disease; central retinal vein occlusion; uveitic retinal disease; retinal detachment; ocular trauma which affects a posterior ocular site or location; a posterior ocular condition caused by or influenced by an ocular laser treatment; posterior ocular conditions caused by or influenced by a photodynamic therapy; photocoagulation; radiation retinopathy; epiretinal membrane disorders; branch retinal vein occlusion; anterior ischemic optic neuropathy; non-retinopathy diabetic retinal dysfunction, retinitis pigmentosa and glaucoma. Glaucoma can be considered a posterior ocular condition because the therapeutic goal is to prevent the loss of or reduce the occurrence of loss of vision due to damage to or loss of retinal cells or optic nerve cells (i.e. neuroprotection).

An anterior ocular condition can include a disease, ailment or condition, such as for example, aphakia; pseudophakia; astigmatism; blepharospasm; cataract; conjunctival diseases; conjunctivitis; corneal diseases;, corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

The present invention is concerned with and directed to a drug delivery system and methods for the treatment of an ocular condition, such as an anterior ocular condition or a posterior ocular condition or to an ocular condition which can be characterized as both an anterior ocular condition and a posterior ocular condition.

Therapeutic compounds useful for the treatment of an ocular condition can include active agents with, for example, antibiotic, anti-neoplastic, anti-angiogenesis, kinase inhibition, anticholinergic, anti-adrenergic and/or anti-inflammatory activity.

Mitochondrial permeability transition (“MPT”) is an increase in the permeability of the mitochondrial membranes which can result from opening of mitochondrial permeability transition pores (“MPTP”). It is believed that a MPT pore is a protein pore made by mitochondrial membranes during certain disease or conditions such as stroke, cerebral trauma, ischemia, heart attack, Reye's syndrome. Formation of a MPTP can cause mitochondria to swell followed by death of the cell. Mitochondrial permeability transition can be associated with ocular diseases and conditions. See eg Gawrylewski, A., Mitochondrial death throes, The Scientist 21(4): 73; April 2007. Therefore, there is a need for delivering to the eye agents which can stabilize mitochondrial membranes, and thereby treat the eye.

Solid pharmaceutically active implants that provide sustained release of an active ingredient are able to provide a relatively uniform concentration of active ingredients in the body. Implants are particularly useful for providing a high local concentration at a particular target site for extended periods of time. These sustained or extended release forms reduce the number of doses of the drug to be administered, and avoid the peaks and troughs of drug concentration found with traditional drug therapies. Use of a biodegradable drug delivery system has the further benefit that the spent implant need not be removed from the target site. Use of a biodegradable drug delivery system which provides local drug delivery can also minimize or reduce drug systemic concentrations and side effects.

Many of the anticipated benefits of delayed release implants are dependent upon sustained release at a relatively constant level. However, formulations of hydrophobic drugs with biodegradable matrices may have a release profile which shows little or no release until erosion of the matrix occurs, at which point there is a dumping (burst release) of drug.

The eye is of particular interest when formulating implantable drugs, because one can reduce the amount of surgical manipulation required, and provide effective levels of the drug specifically to the eye. When a solution is injected directly into the eye, the drug quickly washes out or is depleted from within the eye into the general circulation. From the therapeutic standpoint, this may be as useless as giving no drug at all. Because of this inherent difficulty of delivering drugs into the eye, successful medical treatment of ocular diseases is inadequate.

Improved sustained release formulations which allow for a constant drug release rate are of considerable interest for medical and veterinary uses. Thus there is a need for an intraocular drug delivery system for treating retinal diseases by stabilizing retinal cell mitochondrial membranes to thereby improve vision.

### SUMMARY

The present invention meets these and other needs and provides an intraocular drug delivery system for treating retinal diseases by stabilizing retinal cell mitochondrial membranes to thereby improve vision.

Definitions

The following terms used herein have the meanings set forth below.

“About” means approximately or nearly and in the context of a numerical value or range set forth herein means ±10% of the numerical value or range recited or claimed.

“Active agent” and “drug” are used interchangeably and refer to any substance used to treat an ocular condition.

“Bioerodible polymer” means a polymer which degrades in vivo, and wherein erosion of the polymer over time is required to achieve the active agent release kinetics according to the present invention. Bioerodible polymer also includes a hydrogel which act to release drug through polymer swelling.

“Cumulative release profile” means to the cumulative total percent of an active agent released from an implant into an ocular region or site in vivo over time or into a specific release medium in vitro over time.

“Extended release” means release of an active therapeutic agent (such as cyclosporine) from a biodegradable polymeric matrix in vitro or in vivo over of period of between about 1 hour and about 1 week.

“Glaucoma” means primary, secondary and/or congenital glaucoma. Primary glaucoma can include open angle and closed angle glaucoma. Secondary glaucoma can occur as a complication of a variety of other conditions, such as injury, inflammation, vascular disease and diabetes.

“Inflammation-mediated” in relation to an ocular condition means any condition of the eye which can benefit from treatment with an anti-inflammatory agent, and is meant to include, but is not limited to, uveitis, macular edema, acute macular degeneration, retinal detachment, ocular tumors, fungal or viral infections, multifocal choroiditis, diabetic uveitis, proliferative vitreoretinopathy (PVR), sympathetic opthalmia, Vogt Koyanagi-Harada (VKH) syndrome, histoplasmosis, and uveal diffusion.

“Injury” or “damage” are interchangeable and refer to the cellular and morphological manifestations and symptoms resulting from an inflammatory-mediated condition, such as, for example, inflammation.

“Measured under infinite sink conditions in vitro,” means assays to measure drug release in vitro, wherein the experiment is designed such that the drug concentration in the receptor medium never exceeds 10% of saturation. Examples of suitable assays may be found, for example, in USP 23; NF 18 (1995) pp. 1790-1798.

“Ocular condition” means a disease, aliment or condition which affects or involves the eye or one or the parts or regions of the eye, such as a retinal disease. The eye includes the eyeball and the tissues and fluids which constitute the eyeball, the periocular muscles (such as the oblique and rectus muscles) and the portion of the optic nerve which is within or adjacent to the eyeball.

“Plurality” means two or more.

“Posterior ocular condition” means a disease, ailment or condition which affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerve which vascularize or innervate a posterior ocular region or site.

“Steroidal anti-inflammatory agent” and “glucocorticoid” are used interchangeably herein, and are meant to include steroidal agents, compounds or drugs which reduce inflammation when administered at a therapeutically effective level.

“Sustained release” means release of an active therapeutic agent (such as cyclosporine) from a biodegradable polymeric matrix in vitro or in vivo over of period of time between greater than about 1 week and up to about 1 year.

“Substantially” in relation to the release profile or the release characteristic of an active agent from a bioerodible implant as in the phrase “substantially continuous rate” of the active agent release rate from the implant means, that the rate of release (i.e. amount of active agent released/unit of time) does not vary by more than 100%, and preferably does not vary by more than 50%, over the period of time selected (i.e. a number of days). “Substantially” in relation to the blending, mixing or dispersing of an active agent in a polymer, as in the phrase “substantially homogenously dispersed” means that the particles of active agent are homogenously mixed and dispersed throughout the polymeric matrix.

“Suitable for insertion (or implantation) in (or into) an ocular region or site” with regard to an implant, means an implant which has a size (dimensions) such that it can be inserted or implanted without causing excessive tissue damage and without unduly physically interfering with the existing vision of the patient into which the implant is implanted or inserted.

“Therapeutic levels” or “therapeutic amount” means an amount or a concentration of an active agent that has been locally delivered to an ocular region that is appropriate to safely treat an ocular condition so as to reduce or prevent a symptom of an ocular condition.

The present invention includes methods of treating ocular diseases and conditions with a therapeutic agent containing biodegradable ocular implant. The treatment methods target mitochondrial permeability transition which is an increase in the permeability of the mitochondrial membranes. Delivery of the therapeutic agent containing, biodegradable ocular implant to a subject can block the formation of the mitochondrial permeability transition pore, thereby stabilizing mitochondrial membranes, leading thence to vision improvement.

One embodiment of the present invention is methods of treating ocular diseases and conditions comprising stabilizing mitochondrial membranes by blocking the formation of mitochondrial permeability transition pores, the stabilizing comprising implanting into a mammal a biodegradable ocular implant comprising cyclosporine and a biodegradable polymer, wherein the biodegradable polymer is polylactic acid polyglycolic acid copolymer, and the implant is formed for subconjunctival insertion. Preferably the cyclosporin used is cyclosporin-A. More preferably the cyclosporin-A used contains less than 20 ppm heavy metals, no more than 0.7 wt % cyclosporine G, no more than 0.5 wt % cyclosporine B, and no more than 0.3 wt % cyclosporine C.

The ocular disease or condition treated can be a retinal disease or condition. The DDS can comprise a polylactic acid polyglycolic acid copolymer present in an amount of at least 20 weight percent of the total implant (DDS) weight. The active agent in the DDS can be a cyclosporine which is dispersed within the polylactic acid polyglycolic acid copolymer. The implant can be made by an extrusion method. The implant can be in the form of a sheets, particle, fiber, microcapsule, disc, microsphere or filament (rod shaped).

The implant can comprise a plurality of particles or microspheres. The active agent can be homogeneously distributed through the biodegradable polymer. The implant is dimensioned to be compatible with the size and shape of the site of insertion, for example so as to facilitate subconjunctival, sub-tenon or intravitreal insertion of the implant. The cyclosporine is present in the implant an amount in a range of at least about 1 weight percent to about 95 weight percent of the implant. The biodegradable ocular implant can further comprises at least one additional therapeutic agent and/or a release modulator.

The present invention includes a method of treating an ocular disease or condition by stabilizing mitochondrial membranes by blocking the formation of mitochondrial permeability transition pores, said stabilizing comprising implanting into a mammal a biodegradable ocular implant comprising a MPT pore blocker, such as a cyclosporine, and a carrier for the cyclosporine. The carrier for the cyclosporine can be a biodegradable polymer such as a hydrogel such as a viscous hyaluronic acid (such as a cross linked hyaluronic acid, a non-cross linked hyaluronic acid or a mixture thereof). The biodegradable polymer can also be a polylactic acid polyglycolic acid copolymer. The implant is formed for intraocular insertion. The polymer can be in the form of microspheres (and the microspheres can be combined with a hyaluronic acid carrier, thereby providing a formulation with low immunogenicity which can be administered by syringe)) or the implant can be in the form of a single larger (monolithic) implant. The implant can in vivo release between about 1% to about 100% of the cyclosporine over a period of between about 1 day and 1 year. For example, an implant within the scope of the present invention can in vivo release between about 20% to about 90% of the cyclosporine over a period of between about 5 days and 6 months or between about 60% to about 100% of the cyclosporine over a period of between about 1 week and 4 months, or between about 70% to about 100% of the cyclosporine over a period of between 25 days to 60 days. The present invention also includes a method for treating an ocular disease or condition, the method comprising significant stabilization of retinal cell mitochondrial membranes by blocking the formation of mitochondrial permeability transition pores, said stabilizing comprising implanting into a human eye a biodegradable ocular implant comprising cyclosporine and a biodegradable polymer, wherein said biodegradable polymer is a polylactic acid polyglycolic acid copolymer, and said implant is formed for intravitreal insertion.

Finally, the present invention also includes a drug delivery system for treating an ocular disease or condition, the system comprising a cyclosporine for stabilizing posterior ocular mitochondrial membranes by blocking the formation of mitochondrial permeability transition pores, and a biodegradable polylactic acid polyglycolic acid copolymer, said system being formed for intraocular insertion.

### DRAWINGS

FIG. 1 is a diagram showing a cross-sectional view of a human eye.

FIG. 2 is a graph showing on the x axis time in days after placement of a cyclosporine DDS (implant) into an in vitro assay system buffer, and on the y axis cumulative total amount of cyclosporine release from the DDS into the buffer solution. The six different implant in vitro release profiles shows in FIG. 2 are for selected Table 1 implants made as set forth in Example 1.

### DESCRIPTION

The implants of the present invention can include an active agent mixed with or dispersed within a biodegradable polymer. The implant compositions can vary according to the preferred drug release profile, the particular active agent used, the ocular condition being treated, and the medical history of the patient. Active agents that may be used include, but are not limited to (either by itself in an implant within the scope of the present invention or in combination with another active agent): ace-inhibitors, endogenous cytokines, agents that influence basement membrane, agents that influence the growth of endothelial cells, adrenergic agonists or blockers, cholinergic agonists or blockers, aldose reductase inhibitors, analgesics, anesthetics, antiallergics, anti-inflammatory agents, antihypertensives, pressors, antibacterials, antivirals, antifungals, antiprotozoals, anti-infectives, antitumor agents, antimetabolites, antiangiogenic agents, tyrosine kinase inhibitors, antibiotics such as aminoglycosides such as gentamycin, kanamycin, neomycin, and vancomycin; amphenicols such as chloramphenicol; cephalosporins, such as cefazolin HCl; penicillins such as ampicillin, penicillin, carbenicillin, oxycillin, methicillin; lincosamides such as lincomycin; polypeptide antibiotics such as polymixin and bacitracin; tetracyclines such as tetracycline; quinolones such as ciproflaxin, etc.; sulfonamides such as chloramine T; and sulfones such as sulfanilic acid as the hydrophilic entity, anti-viral drugs, e.g. acyclovir, gancyclovir, vidarabine, azidothymidine, dideoxyinosine, dideoxycytosine, dexamethasone , ciproflaxin, water soluble antibiotics, such as acyclovir, gancyclovir, vidarabine, azidothymidine, dideoxyinosine, dideoxycytosine; epinephrine; isoflurphate; adriamycin; bleomycin; mitomycin; ara-C; actinomycin D; scopolamine; and the like, analgesics, such as codeine, morphine, keterolac, naproxen, etc., an anesthetic, e.g. lidocaine; .beta.-adrenergic blocker or .beta.-adrenergic agonist, e.g. ephidrine, epinephrine, etc.; aldose reductase inhibitor, e.g. epalrestat, ponalrestat, sorbinil, tolrestat; antiallergic, e.g. cromolyn, beclomethasone, dexamethasone, and flunisolide; colchicine, anihelminthic agents, e.g. ivermectin and suramin sodium; antiamebic agents, e.g. chloroquine and chlortetracycline; and antifungal agents, e.g. amphotericin, etc., anti-angiogenesis compounds such as anecortave acetate, retinoids such as Tazarotene, anti-glaucoma agents, such as brimonidine (Alphagan and Alphagan P), acetozolamide, bimatoprost (Lumigan), timolol, mebefunolol; memantine; alpha-2 adrenergic receptor agonists; 2-methoxyestradiol; anti-neoplastics, such as vinblastine, vincristine, interferons; alpha, beta and gamma., antimetabolites, such as folic acid analogs, purine analogs, and pyrimidine analogs; immunosuppressants such as azathiprine, cyclosporine and mizoribine; miotic agents, such as carbachol, mydriatic agents such as atropine, etc., protease inhibitors such as aprotinin, camostat, gabexate, vasodilators such as bradykinin, etc., and various growth factors, such epidermal growth factor, basic fibroblast growth factor, nerve growth factors, and the like.

In one variation the active agent is methotrexate. In another variation, the active agent is a retinoic acid. In another variation, the active agent is an anti-inflammatory agent such as a nonsteroidal anti-inflammatory agent. Nonsteroidal anti-inflammatory agents that may be used include, but are not limited to, aspirin, diclofenac, flurbiprofen, ibuprofen, ketorolac, naproxen, and suprofen. In a further variation, the anti-inflammatory agent is a steroidal anti-inflammatory agent, such as dexamethasone.

The steroidal anti-inflammatory agents that may be used in the ocular implants include, but are not limited to, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, and any of their derivatives.

In one embodiment, cortisone, dexamethasone, fluocinolone, hydrocortisone, methylprednisolone, prednisolone, prednisone, and triamcinolone, and their derivatives, are preferred steroidal anti-inflammatory agents. In another preferred variation, the steroidal anti-inflammatory agent is dexamethasone. In another variation, the biodegradable implant includes a combination of two or more steroidal anti-inflammatory agents.

The active agent, such as a cyclosporin, can comprise from about 10% to about 90% by weight of the implant. In one variation, the agent is from about 40% to about 80% by weight of the implant. In one embodiment, the agent comprises about 60% by weight of the implant. In another embodiment of the present invention, the agent can comprise about 50% by weight of the implant.

In one variation, the active agent can be homogeneously dispersed in the biodegradable polymer of the implant. The implant can be made, for example, by a sequential or double extrusion method. The selection of the biodegradable polymer used can vary with the desired release kinetics, patient tolerance, the nature of the disease to be treated, and the like. Polymer characteristics that are considered include, but are not limited to, the biocompatibility and biodegradability at the site of implantation, compatibility with the active agent of interest, and processing temperatures. The biodegradable polymer matrix usually comprises at least about 10, at least about 20, at least about 30, at least about 40, at least about 50, at least about 60, at least about 70, at least about 80, or at least about 90 weight percent of the implant. In one variation, the biodegradable polymer matrix comprises about 40% to 50% by weight of the implant.

Biodegradable polymers which can be used include, but are not limited to, polymers made of monomers such as organic esters or ethers, which when degraded result in physiologically acceptable degradation products. Anhydrides, amides, orthoesters, or the like, by themselves or in combination with other monomers, may also be used. The polymers are generally condensation polymers. The polymers can be cross-linked or noncross-linked. If cross-linked, they are usually not more than lightly cross-linked, and are less than 5% cross-linked, usually less than 1% cross-linked.

For the most part, besides carbon and hydrogen, the polymers will include oxygen and nitrogen, particularly oxygen. The oxygen may be present as oxy, e.g., hydroxy or ether, carbonyl, e.g., non-oxo-carbonyl, such as carboxylic acid ester, and the like. The nitrogen can be present as amide, cyano, and amino. An exemplary list of biodegradable polymers that can be used are described in Heller, Biodegradable Polymers in Controlled Drug Delivery, In: “CRC Critical Reviews in Therapeutic Drug Carrier Systems”, Vol. 1. CRC Press, Boca Raton, Fla. (1987).

Of particular interest are polymers of hydroxyaliphatic carboxylic acids, either homo- or copolymers, and polysaccharides. Included among the polyesters of interest are homo- or copolymers of D-lactic acid, L-lactic acid, racemic lactic acid, glycolic acid, caprolactone, and combinations thereof. Copolymers of glycolic and lactic acid are of particular interest, where the rate of biodegradation is controlled by the ratio of glycolic to lactic acid. The percent of each monomer in poly(lactic-co-glycolic)acid (PLGA) copolymer may be 0-100%, about 15-85%, about 25-75%, or about 35-65%. In certain variations, 25/75 PLGA and/or 50/50 PLGA and/or 75/25 PLGA, and/or 85/15 PLGA copolymers are used. In other variations, PLGA copolymers are used in conjunction with polylactide polymers.

Biodegradable polymer matrices that include mixtures of hydrophilic and hydrophobic ended PLGA may also be employed, and are useful in modulating polymer matrix degradation rates. Hydrophobic ended (also referred to as capped or end-capped) PLGA has an ester linkage hydrophobic in nature at the polymer terminus. Typical hydrophobic end groups include, but are not limited to alkyl esters and aromatic esters. Hydrophilic ended (also referred to as uncapped) PLGA has an end group hydrophilic in nature at the polymer terminus. PLGA with a hydrophilic end groups at the polymer terminus degrades faster than hydrophobic ended PLGA because it takes up water and undergoes hydrolysis at a faster rate (Tracy et al., Biomaterials 20:1057-1062 (1999)). Examples of suitable hydrophilic end groups that may be incorporated to enhance hydrolysis include, but are not limited to, carboxyl, hydroxyl, and polyethylene glycol. The specific end group will typically result from the initiator employed in the polymerization process. For example, if the initiator is water or carboxylic acid, the resulting end groups will be carboxyl and hydroxyl. Similarly, if the initiator is a monofunctional alcohol, the resulting end groups will be ester or hydroxyl.

The implants can be formulated with different polymer blends, or of similar blends but with different excipients, and are designed to erode at different rates in situ. The present invention offers the formulator additional degrees of freedom, thereby facilitating extended release for as long as three to six months while avoiding high drug loading and excessive burst release of very water-soluble drugs.

Excipients that may be incorporated into the implants can include poorly water-soluble molecules such as long chain fatty alcohols, cholesterol, or high molecular weight polyethylene glycol polymers. These excipients may fill voids and pores in the polymer matrix and retard undesirable burst release of water-soluble drugs. Concentrations of certain excipients in the implant can dramatically slow drug release rates, an effect which is advantageous for designing optimum sustained release kinetics.

Other agents may be employed in the formulation of an implant within the scope of the present invention for a variety of purposes. For example, buffering agents and preservatives may be employed. Preservatives which may be used include, but are not limited to, sodium bisulfite, sodium bisulfate, sodium thiosulfate, benzalkonium chloride, chlorobutanol, thimerosal, phenylmercuric acetate, phenylmercuric nitrate, methylparaben, polyvinyl alcohol and phenylethyl alcohol. Examples of buffering agents that may be employed include, but are not limited to, sodium carbonate, sodium borate, sodium phosphate, sodium acetate, sodium bicarbonate, and the like, as approved by the FDA for the desired route of administration. Electrolytes such as sodium chloride and potassium chloride may also be included in the formulation.

The biodegradable ocular implants can also include additional hydrophilic or hydrophobic compounds that accelerate or retard release of the active agent. Additionally, release modulators such as those described in U.S. Pat. No. 5,869,079 can be included in the implants. The amount of release modulator employed will be dependent on the desired release profile, the activity of the modulator, and on the release profile of the glucocorticoid in the absence of modulator. Where the buffering agent or release enhancer or modulator is hydrophilic, it may also act as a release accelerator. Hydrophilic additives act to increase the release rates through faster dissolution of the material surrounding the drug particles, which increases the surface area of the drug exposed, thereby increasing the rate of drug diffusion. Similarly, a hydrophobic buffering agent or enhancer or modulator can dissolve more slowly, slowing the exposure of drug particles, and thereby slowing the rate of drug diffusion.

An implant within the scope of the present invention can be formulated with particles of an active agent dispersed within a biodegradable polymer matrix. Without being bound by theory, it is believed that the release of the active agent can be achieved by hydrolysis of the biodegradable polymer matrix and by diffusion of the active agent into an ocular fluid, e.g., the vitreous, with subsequent erosion of the polymer matrix and release of the active agent. Factors which influence the release kinetics of active agent from the implant can include such characteristics as the size and shape of the implant, the size of the active agent particles, the solubility of the active agent, the ratio of active agent to polymer(s), the method of manufacture, the surface area, and the hydrolysis rate of the polymer(s). The release kinetics achieved by this form of active agent release are different than that achieved through formulations which release active agents through polymer swelling, such as with cross-linked hydrogels. In that case, the active agent is not released through polymer degradation, but through polymer swelling and drug diffusion, which releases agent as liquid diffuses through the pathways exposed.

The release rate of the active agent can depend at least in part on the rate of degradation of the polymer backbone component or components making up the biodegradable polymer matrix. For example, condensation polymers may be degraded by hydrolysis (among other mechanisms) and therefore any change in the composition of the implant that enhances water uptake by the implant will likely increase the rate of hydrolysis, thereby increasing the rate of polymer degradation and erosion, and thus increasing the rate of active agent release.

The release kinetics of the implants of the present invention can be dependent in part on the surface area of the implants. A larger surface area exposes more polymer and active agent to ocular fluid, causing faster degradation of the polymer matrix and dissolution of the active agent particles in the matrix. Therefore, the size and shape of the implant may also be used to control the rate of release, period of treatment, and active agent concentration at the site of implantation. At equal active agent loads, larger implants will deliver a proportionately larger dose, but depending on the surface to mass ratio, may possess a slower release rate based on total percent released. For implantation in an ocular region, the total weight of the implant preferably ranges, e.g., from about 100 μg to about 15 mg. Alternatively, the implant weight ranges from about 300 μg to about 10 mg, or from about 500 μg to about 5 mg. In a particular embodiment of the present invention the weight of an implant is between about 500 μg and about 2 mg, such as between about 500 μg and about 1 mg.

Examples of ocular conditions which can be treated by the implants and methods of the invention include, but are not limited to, glaucoma, uveitis, macular edema, macular degeneration, retinal detachment, posterior ocular tumors, fungal or viral infections, multifocal choroiditis, diabetic retinopathy, proliferative vitreoretinopathy (PVR), sympathetic opthalmia, Vogt Koyanagi-Harada (VKH) syndrome, histoplasmosis, uveal diffusion, and vascular occlusion. In one variation, the implants are particularly useful in treating such medical conditions as uveitis, macular edema, vascular occlusive conditions, proliferative vitreoretinopathy (PVR), and various other retinopathies.

The biodegradable implants can be inserted into the eye by a variety of methods, including placement by forceps, by trocar, or by other types of applicators, after making an incision in the sclera. In some instances, a trocar or applicator may be used without creating an incision. In a preferred variation, a hand-held applicator is used to insert one or more biodegradable implants into the eye. The hand-held applicator typically comprises an 18-30 GA stainless steel needle, a lever, an actuator, and a plunger. Suitable devices for inserting an implant or implants into a posterior ocular region or site includes those disclosed in U.S. patent application Ser. No. 10/666,872.

The method of implantation generally first involves accessing the target area within the ocular region with the needle, trocar or implantation device. Once within the target area, e.g., the vitreous cavity, a lever on a hand held-device can be depressed to cause an actuator to drive a plunger forward. As the plunger moves forward, it can push the implant or implants into the target area (i.e. the vitreous).

Various techniques may be employed to make implants within the scope of the present invention. Useful techniques include phase separation methods, interfacial methods, extrusion methods, compression methods, molding methods, injection molding methods, heat press methods and the like.

Choice of the technique, and manipulation of the technique parameters employed to produce the implants can influence the release rates of the drug. Room temperature compression methods result in an implant with discrete microparticles of drug and polymer interspersed. Extrusion methods result in implants with a progressively more homogenous dispersion of the drug within a continuous polymer matrix, as the production temperature is increased.

The use of extrusion methods allows for large-scale manufacture of implants and results in implants with a homogeneous dispersion of the drug within the polymer matrix. When using extrusion methods, the polymers and active agents that are chosen are stable at temperatures required for manufacturing, usually at least about 50° C. Extrusion methods use temperatures of about 25° C. to about 150° C., more preferably about 60° C. to about 130° C.

Different extrusion methods may yield implants with different characteristics, including but not limited to the homogeneity of the dispersion of the active agent within the polymer matrix. For example, using a piston extruder, a single screw extruder, and a twin screw extruder will generally produce implants with progressively more homogeneous dispersion of the active. When using one extrusion method, extrusion parameters such as temperature, extrusion speed, die geometry, and die surface finish will have an effect on the release profile of the implants produced.

In one variation of producing implants by a piston extrusion methods, the drug and polymer are first mixed at room temperature and then heated to a temperature range of about 60° C. to about 150° C., more usually to about 100° C. for a time period of about 0 to about 1 hour, more usually from about 0 to about 30 minutes, more usually still from about 5 minutes to about 15 minutes, and most usually for about 10 minutes. The implants are then extruded at a temperature of about 60° C. to about 130° C., preferably at a temperature of about 90° C.

In an exemplary screw extrusion method, the powder blend of active agent and polymer is added to a single or twin screw extruder preset at a temperature of about 80° C. to about 130° C., and directly extruded as a filament or rod with minimal residence time in the extruder. The extruded filament or rod is then cut into small implants having the loading dose of active agent appropriate to treat the medical condition of its intended use.

Implant systems according to the present invention can be fast release implants made of certain lower molecular weight, fast degradation profile polylactide polymers, such as R104 made by Boehringer Ingelheim GmbH, Germany, which is a poly(D,L-lactide) with a molecular weight of about 3,500. Examples of medium rate release implants include those made of certain medium molecular weight, intermediate degradation profile PLGA co-polymers, such as RG755 made by Boehringer Ingelheim GmbH, Germany, which is a poly(D,L-lactide-co-glycolide with wt/wt 75% lactide :25% glycolide, a molecular weight of about 40,000 and an inherent viscosity of 0.50 to 0.70 dl/g, in 1% chloroform at 25° C. Examples of slow release implants include those made of certain other high molecular weight, slower degradation profile polylactide polymers, such as R203/RG755 made by Boehringer Ingelheim GmbH, Germany, for which the molecular weight is about 14,000 for R203 (inherent viscosity of 0.25 to 0.35 dl/g, in 1%, chloroform at 25° C.) and about 40,000 for RG755.

Individual bioerodible implants with extended or variable release profiles can also be prepared according to the invention using two or more different bioerodible polymers each having different release characteristics. In one such method, particles of a drug or active agent are blended with a first polymer and extruded to form a filament or rod. This filament or rod is then itself broken first into small pieces and then further ground into particles with a size (diameter) between about 30 μm and about 50 μm. which are then blended with an additional quantities of the drug or active agent and a second polymer. This second mixture is then extruded into filaments or rods which are then cut to the appropriate size to form the final implant. The resultant implant has a release profile different than that of an implant created by initially blending the two polymers together and then extruding it. It is believed that formed implants include initial particles of the drug and first polymer having certain specific release characteristics bound up in the second polymer and drug blend that itself has specific release characteristics that are distinct from the first.

Examples of implants include those formed with RG755, R203, RG503, RG502, RG 502H as the first polymer, and RG502, RG 502H as the second polymer. Other polymers that can be used include PDL (poly(D,L-lactide)) and PDLG (poly(D,L-lactide-co-glycolide)) polymers available from PURAC America, Inc. Lincolnshire, Ill. Poly(caprolactone) polymers can also be used. The characteristics of the specified polymers are (1) RG755 has a molecular weight of about 40,000, a lactide content (by weight) of 75%, and a glycolide content (by weight) of 25%; (2) R203 has a molecular weight of about 14,000, and a lactide content of 100% ; (3) RG503 has a molecular weight of about 28,000, a lactide content of 50%, and a glycolide content of 50%; (4) RG502 has a molecular weight of about 11,700 (inherent viscosity of 0.16 to 0.24 dl/g, in 1% chloroform at 25° C.), a lactide content of 50%, and a glycolide content of 50%, and; (5) RG502 H has a molecular weight of about 8,500, a lactide content of 50%, a glycolide content of 50% and free acid at the end of polymer chain.

Generally, if inherent viscosity is 0.16 the molecular weight is about 6,300, and if the inherent viscosity is 0.28 the molecular weight is about 20,700. It is important to note that all polymer molecular weights set forth herein are averaged molecular weights in Daltons. Additionally, the molecular weight depends on the conditions of the measurement, a calibration curve is required to determine molecular weight and the molecular weight so determined is valid only for that class of polymers.

Cyclosporine can be delivered into the eye with sustained release biodegradable implants which can treat ocular diseases and conditions. According to the present disclosure, this is done by blocking the formation of the mitochondrial permeability transition pore. Cyclosporine interacts with cyclophilin from the mitochondrial matrix to prevent its joining the pore thus stabilizing the mitochondrial membranes.

One embodiment of the present disclosure relates to methods of treating ocular diseases and conditions comprising stabilizing mitochondrial membranes by blocking the formation of mitochondrial permeability transition pores, the stabilizing comprising implanting into a mammal a therapeutic agent containing biodegradable ocular implant comprising cyclosporine and a biodegradable polymer, wherein the biodegradable polymer is polylactic acid polyglycolic acid copolymer, and the implant is formed for subconjunctival insertion.

Ocular diseases and conditions as recited herein include retinal diseases and conditions. Retinal diseases and conditions are those of the retina which is a thin layer of neural cells that lines the back of the eyeball of vertebrates and some cephalopods. It is comparable to the film in a camera. In vertebrate embryonic development, the retina and the optic nerve originate as outgrowths of the developing brain. Hence, the retina is part of the central nervous system (CNS). It is the only part of the CNS that can be imaged directly.

A controlled drug release is achieved by the present improved formulation of slow release biodegradable ocular implants. The release rate of a therapeutic agent, an example of which is cyclosporine, from an implant is modulated by addition of a release modulator to the implant. Release of a hydrophobic agent is increased by inclusion of an accelerator in the implant, while retardants are included to decrease the release rate of hydrophilic agents. The release modulator may be physiologically inert, or a therapeutic agent.

The rate of release of the therapeutic agent will be controlled by the rate of transport through the polymeric matrix of the implant, and the action of the modulator. By modulating the release rate, the agent is released at a substantially constant rate, or within a therapeutic dosage range, over the desired period of time. The rate of release will usually not vary by more than about 100% over the desired period of time, more usually by not more than about 50%. The agent is made available to the specific site(s) where the agent is needed, and it is maintained at an effective dosage. The transport of drug through the polymer barrier will also be affected by drug solubility, polymer hydrophilicity, extent of polymer cross-linking, expansion of the polymer upon water absorption so as to make the polymer more permeable to the drug, geometry of the implant, and the like.

The release modulator is an agent that alters the release of a drug from a biodegradable implant in a defined manner. It may be an accelerator or a retardant. Accelerators will be hydrophilic compounds, which are used in combination with hydrophobic agents to increase the rate of release. Hydrophilic agents are those compounds which have at least about 100 μg/mL solubility in water at ambient temperature. Hydrophobic agents are those compounds which have less than about 100 μg/ml solubility in water at ambient temperature. Modulators can also act by changing the glass transition temperature of the polymer or plasticizing the polymers. Additionally, excipients can be added that may catalyze or retard polymer erosion and thereby affect release rates.

Therapeutically active hydrophobic agents which benefit from release modulation include cyclosporines, e.g. cyclosporin A, cyclosporin G, etc.; vinca alkaloids, e.g. vincristine and vinblastine; methotrexate; retinoic acid; certain antibiotics, e.g. ansamycins such as rifampin; nitrofurans such as nifuroxazide; non-steroidal antiinflammatory drugs, e.g. diclofenac, keterolac, flurbiprofen, naproxen, suprofen, ibuprofen, aspirin, etc. Steroids are of particular interest, including hydrocortisone, cortisone, prednisolone, prednisone, dexamethasone, medrysone, fluorometholone, estrogens, progesterones, etc.

Accelerators may be physiologically inert, water soluble polymers, e.g. low molecular weight methyl cellulose or hydroxypropyl methyl cellulose (HPMC); sugars, e.g. monosaccharides such as fructose and glucose, disaccharides such as lactose, sucrose, or polysaccharides such as cellulose, amylose, dextran, etc. Alternatively, the accelerator may be a physiologically active agent, allowing for a combined therapeutic formulation. The choice of accelerator in such a case will be determined by the desired combination of therapeutic activities.

Formulations of particular interest will have a therapeutic combination of two or more active agents, which provides for a sustained release of the agents. Combinations may include steroids, as indicated above, as the hydrophobic agent and water soluble antibiotics, e.g. aminoglycosides such as gentamycin, kanamycin, neomycin, and vancomycin; amphenicols such as chloramphenicol; cephalosporins, such as cefazolin HCl; penicillins such as ampicillin, penicillin, carbenicillin, oxycillin, methicillin; lincosamides such as lincomycin; polypeptide antibiotics such as polymixin and bacitracin; tetracyclines such as tetracycline; quinolones such as ciproflaxin, etc.; sulfonamides such as chloramine T; and sulfones such as sulfanilic acid as the hydrophilic entity. A combination of non-steroidal anti-inflammatory drugs, as indicated above, with water soluble antibiotics is also of interest. Combinations of anti-viral drugs, e.g. acyclovir, gancyclovir, vidarabine, azidothymidine, dideoxyinosine, dideoxycytosine with steroidal or non-steroidal anti-inflammatory drugs, as indicated above, are of interest. A particular combination of interest is dexamethasone and ciproflaxin.

Release retardants are hydrophobic compounds which slow the rate of release of hydrophilic drugs, allowing for a more extended release profile. Hydrophilic drugs of interest which may benefit from release modulation include water soluble antibiotics, as described above, nucleotide analogs, e.g. acyclovir, gancyclovir, vidarabine, azidothymidine, dideoxyinosine, dideoxycytosine; epinephrine; isoflurphate; adriamycin; bleomycin; mitomycin; ara-C; actinomycin D; scopolamine; and the like.

Agents of interest as release retardants include non-water soluble polymers, e.g. high molecular weight methylcellulose and ethylcellulose, etc., low water soluble organic compounds, and pharmaceutically active hydrophobic agents, as previously described.

A combined and-inflammatory drug, and antibiotic or antiviral, may be further combined with an additional therapeutic agent. The additional agent may be an analgesic, e.g. codeine, morphine, keterolac, naproxen, etc., an anesthetic, e.g. lidocaine; β adrenergic blocker or β adrenergic agonist, e.g. ephidrine, epinephrine, etc.; aldose reductase inhibitor, e.g. epalrestat, ponalrestat, sorbinil, tolrestat; antiallergic, e.g. cromolyn, beclomethasone, dexamethasone, and flunisolide; colchicine. Anihelminthic agents, e.g. ivermectin and suramin sodium; antiamebic agents, e.g. chloroquine and chlortetracycline; and antifungal agents, e.g. amphotericin, etc. may be co-formulated with an antibiotic and an anti-inflammatory drug. For intra-ocular use, anti-glaucomas agents, e.g. acetozolamide, befunolol, etc. in combinations with and- inflammatory and antimicrobial agents are of interest. For the treatment of neoplasia, combinations with anti-neoplastics, particularly vinblastine, vincristine, interferons α,β and γ, antimetabolites, e.g. folic acid analogs, purine analogs, pyrimidine analogs may be used. Immunosuppressants such as azathiprine, cyclosporine and mizoribine are of interest in combinations. Also useful combinations include mimic agents, e.g. carbachol, mydriatic agents such as atropine, etc., protease inhibitors such as aprotinin, camostat, gabexate, vasodilators such as bradykinin, etc., and various growth factors, such epidermal growth factor, basic fibroblast growth factor, nerve growth factors, and the like.

The amount of active agent employed in the implant, individually or in combination, will vary widely depending on the effective dosage required and rate of release from the implant. Usually the agent will be at least about 1, more usually at least about 10 weight percent of the implant, and usually not more than about 80, more usually not more than about 40 weight percent of the implant. The amount of release modulator employed will be dependent on the desired release profile, the activity of the modulator, and on the release profile of the active agent in the absence of modulator. An agent that is released very slowly or very quickly will require relatively high amounts of modulator. Generally the modulator will be at least 10, more usually at least about 20 weight percent of the implant, and usually not more than about 50, more usually not more than about 40 weight percent of the implant.

Where a combination of active agents is to be employed, the desired release profile of each active agent is determined. If necessary, a physiologically inert modulator is added to precisely control the release profile. The drug release will provide a therapeutic level of each active agent.

The exact proportion of modulator and active agent will be empirically determined by formulating several implants having varying amounts of modulator. A USP approved method for dissolution or release test will be used to measure the rate of release (USP 23; NF 18 (1995) pp. 1790-1798). For example, using the infinite sink method, a weighed sample of the drug delivery device is added to a measured volume of a solution containing four parts by weight of ethanol and six parts by weight of deionized water, where the solution volume will be such that the drug concentration is after release is less than 5% of saturation. The mixture is maintained at 37° C. and stirred slowly to maintain the implants in suspension. The appearance of the dissolved drug as a function of time may be followed by various methods known in the art, such as spectrophotometrically, HPLC, mass spectroscopy, etc. until the absorbance becomes constant or until greater than 90% of the drug has been released. The drug concentration after 1 h in the medium is indicative of the amount of free unencapsulated drug in the dose, while the time required for 90% drug to be released is related to the expected duration of action of the dose in vivo. Normally the release will be free of larger fluctuations from some average value which allows for a relatively uniform release, usually following a brief initial phase of rapid release of the drug.

Normally the implant will be formulated to release the active agent(s) over a period of at least about 3 days, more usually at least about one week, and usually not more than about one year, more usually not more than about three months. For the most part, the matrix of the implant will have a physiological lifetime at the site of implantation at least equal to the desired period of administration, preferably at least twice the desired period of administration, and may have lifetimes of 5 to 10 times the desired period of administration. The desired period of release will vary with the condition that is being treated. For example, implants designed for post-cataract surgery will have a release period of from about 3 days to 1 week; treatment of uveitis may require release over a period of about 4 to 6 weeks; while treatment for cytomegalovirus infection may require release over 3 to 6 months, or longer.

The implants are of dimensions commensurate with the size and shape of the region selected as the site of implantation and will not migrate from the insertion site following implantation. The implants will also preferably be at least somewhat flexible so as to facilitate both insertion of the implant at the target site and accommodation of the implant. The implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. Due to ease of manufacture, monolithic implants are usually preferred over encapsulated forms. However, the greater control afforded by the encapsulated, reservoir-type may be of benefit in some circumstances, where the therapeutic level of the drug falls within a narrow window. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment of at least 7 days, preferably greater than two weeks, water insoluble, and the like. The polymer will usually comprise at least about 10, more usually at least about 20 weight percent of the implant.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or noncross-linked, usually not more than lightly cross-linked, generally less than 5%, usually less than 1%. For the most part, besides carbon and hydrogen, the polymers will include oxygen and nitrogen, particularly oxygen. The oxygen may be present as oxy, e.g., hydroxy or ether, carbonyl, e.g., non-oxo-carbonyl, such as carboxylic acid ester, and the like. The nitrogen may be present as amide, cyano and amino. The polymers set forth in Heller, supra, may find use, and that disclosure is specifically incorporated herein by reference.

Of particular interest are polymers of hydroxyaliphatic carboxylic acids, either homo- or copolymers, and polysaccharides. Included among the polyesters of interest are polymers of D lactic acid, L lactic acid, racemic lactic acid, glycolic acid, polycaprolactone, and combinations thereof. By employing the L lactate or D lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries.

Among the polysaccharides will be calcium alginate, and functionalized celluloses, particularly carboxymethylcellulose esters characterized by being water insoluble, a molecular weight of about 5 kD to 500 kD, etc. Biodegradable hydrogels may also be employed in the implants of the subject invention. Hydrogels are typically a copolymer material, characterized by the ability to imbibe a liquid. Exemplary biodegradable hydrogels which may be employed are described in Heller in: Hydrogels in Medicine and Pharmacy, NA. Peppes ed., Vol. III, CRC Press, Boca Raton, Fla., 1987, pp 137-149.

Particles can be prepared where the center may be of one material and the surface have one or more layers of the same or different composition, where the layers may be cross-linked, of different molecular weight, different density or porosity, or the like. For example, the center would comprise a polylactate coated with a polylactate-polyglycolate copolymer, so as to enhance the rate of initial degradation. Most ratios of lactate to glycolate employed will be in the range of about 1:0.1 to 1:1. Alternatively, the center could be polyvinyl alcohol coated with polylactate, so that on degradation of the polylactate the center would dissolve and be rapidly washed out of the implantation site.

The formulation of implants for use in the treatment of ocular conditions, diseases, tumors and disorders are of particular interest. The biodegradable implants may be implanted at various sites, depending on the shape and formulation of the implant, the condition being treated, etc. Suitable sites include the anterior chamber, posterior chamber, vitreous cavity, suprachoroidal space, subconjunctiva, episcleral, intracomeal, epicomeal and sclera. Suitable sites extrinsic to the vitreous comprise the suprachoroidal space; the pars plana and the like. The suprachoroid is a potential space lying between the inner scleral wall and the apposing choroid. Implants that are introduced into the suprachoroid may deliver drugs to the choroid and to the anatomically apposed retina, depending upon the diffusion of the drug from the implant, the concentration of drug comprised in the implant and the like. Implants may be introduced over or into an avascular region. The avascular region may be naturally occurring, such as the pars plana, or a region made to be avascular by surgical methods. Surgically-induced avascular regions may be produced in an eye by methods known in the art such as laser ablation, photocoagulation, cryotherapy, heat coagulation, cauterization and the like. It may be particularly desirable to produce such an avascular region over or near the desired site of treatment, particularly where the desired site of treatment is distant from the pars plana or placement of the implant at the pars plana is not possible. Introduction of implants over an avascular region will allow for diffusion of the drug from the implant and into the inner eye and avoids diffusion of the drug into the bloodstream.

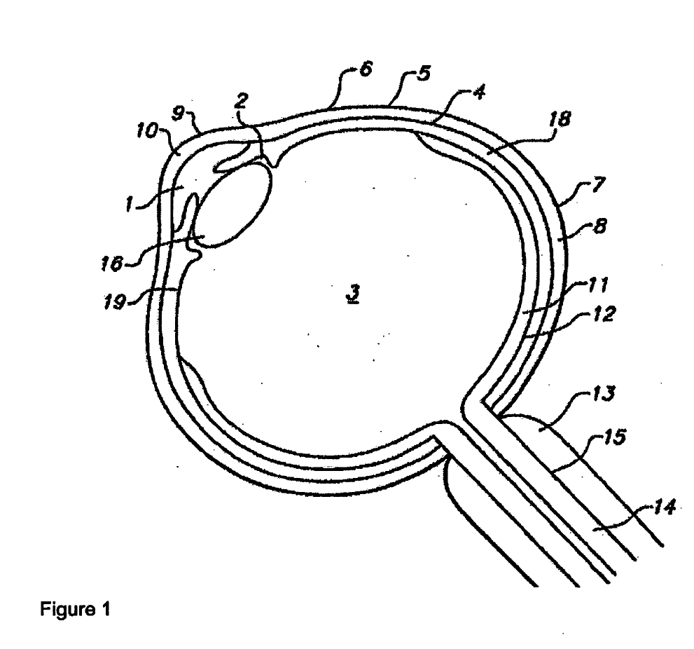

Turning now to FIG. 1, a cross-sectional view of the eye is shown, illustrating the sites for implantation in accordance with the subject invention. The eye comprises a lens 16 and encompasses the vitreous chamber 3. Adjacent to the vitreous chamber 3 is the optic part of the retina 11. Implantation may be intraretinal 11 or subretinal 12. The retina is surrounded by the choroid 18. Implantation may be intrachoroidal or suprachoroidal 4. Between the optic part of the retina and the lens, adjacent to the vitreous, is the pars plana 19. Surrounding the choroid 18 is the sclera 8. Implantation may be intrascleral 8 or episcleral 7. The external surface of the eye is the cornea 9. Implantation may be epicorneal 9 or intra-corneal 10. The internal surface of the eye is the conjunctiva 6. Behind the cornea is the anterior chamber 1, behind which is the lens 16. The posterior chamber 2 surrounds the lens, as shown in the figure. Opposite from the external surface is the optic nerves, and the arteries and vein of the retina. Implants into the meningeal spaces 13, the optic nerve 15 and the intraoptic nerve 14 allows for drug delivery into the central nervous system, and provide a mechanism whereby the blood-brain barrier may be crossed.

Other sites of implantation include the delivery of anti-tumor drugs to neoplastic lesions, e.g. tumor, or lesion area, e.g. surrounding tissues, or in those situations where the tumor mass has been removed, tissue adjacent to the previously removed tumor and/or into the cavity remaining after removal of the tumor. The implants may be administered in a variety of ways, including surgical means, injection, trocar, etc.

Other agents may be employed in the formulation for a variety of purposes. For example, buffering agents and preservatives may be employed. Water soluble preservatives which may be employed include sodium bisulfate, sodium bisulfate, sodium thiosulfate, benzalkonium chloride, chlorobutanol, thimerosal, phenylmercuric acetate, phenylmercuric nitrate, methylparaben, polyvinyl alcohol and phenylethyl alcohol. These agents may be present in individual amounts of from about 0.001 to about 5% by weight and preferably about 0.01 to about 2%. Suitable water soluble buffering agents that may be employed are sodium carbonate, sodium borate, sodium phosphate, sodium acetate, sodium bicarbonate, etc., as approved by the FDA for the desired route of administration. These agents may be present in amounts sufficient to maintain a pH of the system of between 2 to 9 and preferably 4 to 8. As such the buffering agent may be as much as 5% on a weight to weight basis of the total composition. Where the buffering agent or enhancer is hydrophilic, it may also act as a release accelerator, and may replace all or part of the hydrophilic agent. Similarly, a hydrophilic buffering agent or enhance may replace all or part of the hydrophobic agent.

The implants may be of any geometry including fibers, sheets, films, microspheres, circular discs, plaques and the like. The upper limit for the implant size will be determined by factors such as toleration for the implant, size limitations on insertion, ease of handling, etc. Where sheets or films are employed, the sheets or films will be in the range of at least about 0.5 mm×0.5 mm, usually about 3-10 mm×5-10 mm with a thickness of about 0.25-1.0 mm for ease of handling. Where fibers are employed, the diameter of the fiber will generally be in the range of 0.05 to 3 mm. The length of the fiber will generally be in the range of 0.5-10 mm. Spheres will be in the range of 2 μm to 3 mm in diameter.

The size and form of the implant can be used to control the rate of release, period of treatment, and drug concentration at the site of implantation. Larger implants will deliver a proportionately larger dose, but depending on the surface to mass ratio, may have a slower total percentage release rate. The particular size and geometry of an implant will be chosen to best suit the site of implantation. The chambers, e.g. anterior chamber, posterior chamber and vitreous chamber, are able to accommodate relatively large implants of varying geometries, having diameters of 1 to 3 mm. A sheet or circular disk is preferable for implantation in the suprachoroidal space. The restricted space for intraretinal implantation requires relatively small implants, having diameters from 0.5 to 1 mm.

In some situations mixtures of implants may be utilized employing the same or different pharmacological agents. In this way, a cocktail of release profiles, giving a biphasic or triphasic release with a single administration is achieved, where the pattern of release may be greatly varied.

Various techniques may be employed to produce the implants. Useful techniques include solvent evaporation methods, phase separation methods, interfacial methods, extrusion methods, molding methods, injection molding methods, heat press methods and the like. Specific methods are discussed in U.S. Pat. No. 4,997,652, herein incorporated by reference. In a preferred embodiment, extrusion methods are used to avoid the need for solvents in manufacturing. When using extrusion methods, the polymer and drug are chosen so as to be stable at the temperatures required for manufacturing, usually at least about 85° C.

### EXAMPLES

The following examples illustrate aspects of the present invention.

### Example 1

### Methods for Making Cyclosporine Drug Delivery Systems

A bioerodible implant system for extended or sustained delivery of cyclosporine was made by mixing as active agent cyclosporin-A with a biodegradable polymer, as shown in Table 1. In Table 1 “PEG” (polyethylene glycol) is PEG-3350. As shown by Table 1 some of the implants were a blend of two biodegradable polymers (or a biodegradable polymer and another non-polymeric ingredient) and some contained a release modifier such as HPMC (hydroxypropylmethyl cellulose), cholesterol or mannitol. The cyclosporine active agent and the polymer or polymers used were thoroughly mixed at the weight % ratios shown in Table 1 and then fed into a single-piston thermal extruder and extruded cyclosporine-polymer filaments (implants) weighing about 1 mg were thereby made. The cyclosporin-A used was USP grade obtained from Novartis Ringaskiddy Limited (Ireland) sold under the trade name Ciclosporin as product number 150604.

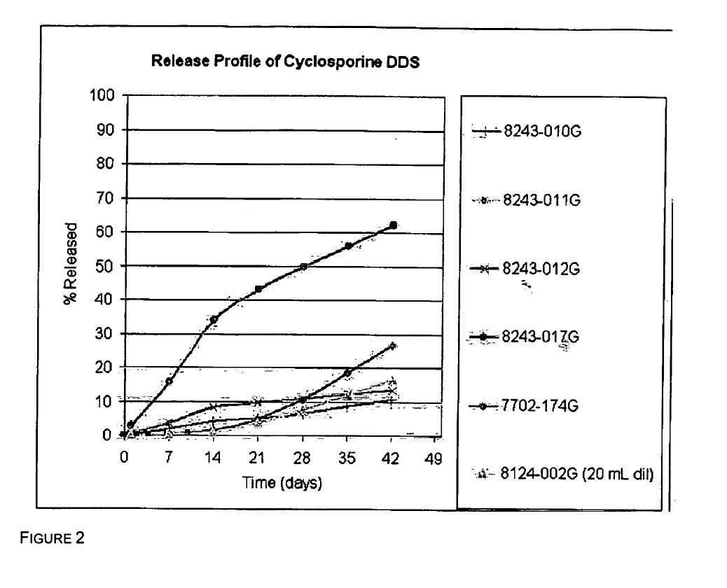

Table 1 shows the specific content of the various cyclosporine formulations made according to this Example 1. In vitro release profiles for six selected Table 1 formulations are shown in FIG. 2. DDS release was measured under infinite sink conditions in vitro using a USP approved method for dissolution or release test for measuring the rate of release (eg USP 23; NF 18 (1995) pp. 1790-1798). Thus the infinite sink method was used in which a weighed sample of the drug delivery implant was added to a measured volume of a solution containing 0.9% NaCl in water, where the solution volume was such that the drug concentration after release was less than 20%, and preferably less than 5%, of saturation. The mixture was maintained at 37° C. and stirred slowly to ensure drug diffusion after bioerosion. The appearance of the dissolved drug as a function of time was followed by various methods known in the art, such as spectrophotometrically, HPLC, mass spectroscopy, etc.

TABLE 1
Cyclosporine DDS Formulations

| | wt % | | | Polymer. or | wt % Polymer. |
|---|---|---|---|---|---|
| Formulation | cyclosporine | Polymer 1 | wt % Polymer 1 | Ingredient 2 | or Ingredient 2 |
| 8124-002G | 40 | RG502H | 60 | | |
| 8124-003G | 30 | RG502H | 60 | PEG | 10 |
| 8124-019G | 50 | RG752S | 50 | | |
| 8124-020G | 30 | RG752S | 70 | | |
| 8124-030G | 40 | R203S | 50 | PEG | 10 |
| 8243-010G | 50 | RG502H | 50 | | |
| 8243-011G | 30 | RG755 | 70 | | |
| 8243-012G | 50 | RG755S | 50 | | |
| 8243-017G | 50 | PEG | 50 | | |
| 7702-174G | 30 | RG502H | 70 | | |
| 8124-002G | 40 | RG502H | 60 | | |
| 8243-013G | 50 | R203S | 50 | | |
| 8243-014g | 30 | RG502H | 60 | HPMC | 10 |
| 8243-015G | 30 | RG502H | 60 | Cholesterol | 10 |
| 8243-016G | 30 | RG502H | 60 | Mannitol | 10 |

### Example 2

### Treatment of an Ocular Condition with a Cyclosporine DDS

An implant made as set forth in Example 1 can be used to treat an ocular condition by implanting the implant into an ocular region or site (i.e. subconjunctival, subtenon, or into the vitreous) of a patient with an ocular condition for a desired therapeutic effect. The ocular condition can be an inflammatory condition such as uveitis or the patient can be afflicted with one or more of the following afflictions: macular degeneration (including non-exudative age related macular degeneration and exudative age related macular degeneration); choroidal neovascularization; acute macular neuroretinopathy; macular edema (including cystoid macular edema and diabetic macular edema); Behcet's disease, diabetic retinopathy (including proliferative diabetic retinopathy); retinal arterial occlusive disease; central retinal vein occlusion; uveitic retinal disease; retinal detachment; retinopathy; an epiretinal membrane disorder; branch retinal vein occlusion; anterior ischemic optic neuropathy; non-retinopathy diabetic retinal dysfunction, retinitis pigmentosa, inherited retinal degeneration (i.e. Best's disease and congenital x-linked retinoschisis), and glaucoma. The implant(s) can be inserted into the vitreous using known procedures (see eg trocar implantation). Alternately, the cyclosporine DDS can be made using one of the methods set forth in and administered to the vitreous using the applicator set forth in Example 8 of U.S. patent application Ser. No. 10/918,597. The implant(s) can release a therapeutic amount of the cyclosporine for an extended or sustained release period of time to thereby treat a symptom of the ocular condition.

The implants made according to Example 1 can be used to treat posterior ocular (i.e. retinal) diseases and conditions by stabilization of mitochondrial membranes in retinal cells. An implant such as number 8243-017G in Table 1 can be used to provide short term drug exposure to retinal cells to treat an acute retinal condition such as a posterior chamber inflammation or retinal detachment, whereas an implant such as number 8243-012G in table 1 can be used provide long term drug exposure to treat a chronic retinal condition such as retinitis pigmentosa, macula edema or macula degeneration.

Stabilization of mitochondrial membranes in retinal cells upon intraocular administration of an Example 1 cyclosporine DDS is evidenced by an improvement in a patient's vision or by a reduction or cessation in the rate of deterioration of the patient's vision (collectively “vision improvement”). A measurement of vision improvement and hence of retinal cell mitochondrial membrane stabilization can be by assessment of the patients' best correct visual acuity (BCVA) as compared to his baseline vision (i.e. just prior to DDS implantation) as measured by the known Early Treatment Diabetic Retinopathy Study (ETDRS) method using periodic patient vision assessments after DDS implantation. For example, according to our invention significant stabilization of retinal cell mitochondrial membranes occurs when at 90 days post implantation of an Example 1 cyclosporine DDS the patient shows a BCVA improvement of at least 2 lines from baseline.

All references, publications, applications and patents cited herein are each incorporated by reference in their entireties. The embodiments of the invention disclosed herein are illustrative of the present invention. Other modifications that may be employed are within the scope of the invention. Thus, by way of example, but not of limitation, alternative configurations of the present invention may be utilized in accordance with the teachings herein. Accordingly, the present invention is not limited to that precisely shown and described.

We claim:

1. A method of treating an ocular disease or condition comprising stabilizing mitochondrial membranes by blocking the formation of mitochondrial permeability transition pores, the stabilizing comprising implanting into a mammal an ocular implant comprising cyclosporine and a polymer, and the implant is formed for intraocular insertion.

2. The method of claim 1 wherein said ocular diseases or conditions are retinal diseases or conditions.

3. The method of claim 1, wherein the polymer is a biodegradable polymer.

4. The method of claim 3, wherein the biodegradable polymer is a polylactic acid polyglycolic acid copolymer.

5. The method of claim 4, wherein the polylactic acid polyglycolic acid copolymer is present in an amount of at least about 20 weight percent of the implant.

6. The method of claim 4, wherein the cyclosporine is dispersed within the polylactic acid polyglycolic acid copolymer.

7. The method of claim 1, wherein the implant is made by an extrusion method.

8. The method of claim 1, wherein the implant includes forms selected from the group consisting of sheets, pluralities of particles, fibers, microcapsules, discs, microspheres and filaments.

9. The method of claim 8, wherein the implant includes forms selected from the group consisting of pluralities of particles and microspheres.

10. The method of claim 1, wherein the implant is dimensioned to be compatible with the size and shape of the site of insertion.

11. The method of claim 1, wherein the implant is structured to facilitate both subconjunctival insertion and accommodation of the implant.

12. The method of claim 1, wherein the cyclosporine is present in an amount in a range of at least about 1 weight percent to about 80 weight percent of the implant.

13. The method of claim 1, wherein the ocular implant further comprises at least one additional therapeutic agent.

14. The method of claim 1, wherein the ocular implant further comprises a release modulator.

15. The method of claim 1, wherein said implant in vivo releases about 60% of the cyclosporine over about 40 days.

16. A method of treating an ocular disease or condition, the method comprising significant stabilization of retinal cell mitochondrial membranes by blocking the formation of mitochondrial permeability transition pores, said stabilizing comprising implanting into a human eye a biodegradable ocular implant comprising cyclosporine and a biodegradable polymer, wherein said biodegradable polymer is a polylactic acid polyglycolic acid copolymer, and said implant is formed for intravitreal insertion.

17. A drug delivery system for treating an ocular disease or condition, the system comprising a cyclosporine for stabilizing posterior ocular mitochondrial membranes by blocking the formation of mitochondrial permeability transition pores, and a biodegradable polylactic acid polyglycolic acid copolymer, said system being formed for intraocular insertion.
